(19) United States
(12) Patent Application Publication
(10) Pub. No.: US 2023/0279478 A1
(43) Pub. Date: Sep. 7, 2023

(54) CASCADED NUCLEIC ACID PROTOCOLS FOR ULTRA-SPECIFIC MOLECULAR DETECTION, TRANSDUCTION, AND AMPLIFICATION

(71) Applicant: DOMUS DIAGNOSTICS, INC., Park City, UT (US)

(72) Inventors: John H. REIF, Durham, NC (US); Xin SONG, Raleigh, NC (US)

(21) Appl. No.: 18/116,138

(22) Filed: Mar. 1, 2023

Related U.S. Application Data
(60) Provisional application No. 63/315,635, filed on Mar. 2, 2022.

(51) Int. Cl. C12Q 1/6848

(52) U.S. Cl. CPC C12Q 1/6848

(57) ABSTRACT

Disclosed herein are designs and implementations of a suite of novel molecular mechanisms that leverage programmable, competitive hybridization and strand displacement of nucleic acids to effectively suppress the generation of nonspecific reaction products (i.e., false positives) in molecular detection, transduction, and isothermal amplification of DNA or RNA targets. Some of the mechanisms described herein may also be applied to enhance the performance of thermocycling-based amplification methods.

### CROSS-REFERENCE TO RELATED APPLICATIONS

This Application claims priority to and the benefit of U.S. Provisional Patent Application No. 63/315,635, filed Mar. 2, 2022, which is incorporated herein by this reference in its entirety.

### BACKGROUND

Nucleic acid sequence amplification is a reaction where the nucleic acid sequence is amplified in quantity. An isothermal reaction is a reaction where there is limited change in temperature. Numerous isothermal amplification techniques have been developed to enable rapid and sensitive detection of nucleic acid sequences without the need for precise temperature cycling as required in conventional polymerase chain reaction (PCR), making it possible to implement low-cost molecular diagnostics for point-of-care and field-deployable applications. State-of-the-art isothermal amplification protocols include loop-mediated isothermal amplification (LAMP), which includes as a subset reverse-transcription LAMP (RT-LAMP).

Additional “LAMP-like” isothermal amplification reactions include other variations of isothermal nucleic acid amplification protocols, such as using dual-priming, swarm priming, stem priming, hairpin primers, etcetera. In general, LAMP and LAMP-like isothermal amplification reactions are prone to false positives partly due to the simultaneous use of multiple primers for target recognition, which increases the likelihood of primer-primer interactions and formation of primer secondary structures leading to non-specific amplification in absence of the target sequence.¹

Nucleic acid hybridization is a technique in which single-stranded nucleic acid sequence and a complementary nucleic acid sequence interact to form a nucleic acid complex. In PCR and LAMP protocols, partial hybridization between the primers and non-target sequences can also lead to spurious amplification. Despite meticulous primer design and in silico optimization efforts, there is still a lack of a universal metric to predict the performance of primers during actual amplification.² As a result, time-consuming experimental screening and manual adjustment of multiple candidate primer sets are typically unavoidable in practice, making the already sophisticated assay design and optimization process even more challenging and tedious.

To improve the assay reliability, prior approaches have made use of chemical or enzymatic additives to delay or prevent the generation of nonspecific amplicons;³ however, these techniques not only increase the cost of the assay but also alter and complicate the overall reaction condition. Other techniques for enhancing the signal-to-noise ratio of isothermal amplification rely on the use of sequence-specific reporter probes or similar constructs to detect the target amplicon.⁴⁻⁸ However, these techniques only distinguish the target amplicon from a resultant pool of amplification products, and cannot prevent the exponential generation of nonspecific amplicons starting from the initial stages of the reaction, and as a result, the assay readout must rely on target-specific detection (e.g., fluorescence-quencher probes). Such methods thus cannot allow the use of more convenient and potentially lower-cost methods (e.g., pH-based colorimetric change, turbidity, intercalating dyes) that directly probe the total DNA synthesis during the amplification. These drawbacks limit the general use of such enhancement techniques in point-of-care assays, which require quick visual interpretation of the test result without reliance on specialized readout devices.

Accordingly, there is an ongoing need for a simple, efficient, and programmable approach for assay specificity enhancement that can be generally applied to different molecular detection, transduction, amplification, and readout protocols.

### SUMMARY

Disclosed herein are compositions and methods directed to novel molecular mechanisms that leverage programmable, competitive hybridization and strand displacement of nucleic acids to effectively suppress the generation of nonspecific reaction products (i.e., false positives) in molecular detection, transduction, and isothermal amplification of DNA or RNA targets. Specifically, the present disclosure proposes a general framework of several molecular mechanisms referred to herein as “armoring/de-armoring”, “thresholding/de-thresholding”, and “deactivating/reactivating”. These molecular mechanisms make use of short single-stranded nucleic acid oligonucleotides (i.e., “oligos”) or pre-annealed oligo dimers that have programmable interactions with the primers, the target sequence, or the transduced strand of the target sequence to control or moderate the initiation and progress of further reactions in different molecular protocols.

Strand displacement is a reaction in a nucleic acid complex that exchanges one nucleic acid strand with another replacement strand. The proposed molecular mechanisms can be designed, modularized, and implemented in vitro based on the well-characterized principles of nucleic acid hybridization and strand displacement.⁹⁻¹² By programming the thermodynamic and kinetic parameters of hybridization and strand displacement, these molecular mechanisms can be custom-programmed and incorporated into different molecular detection, transduction, amplification, and readout pathways to reliably improve the specificity for detecting trace amounts of the target DNA or RNA molecule without sacrificing overall reaction speed and sensitivity.

A reaction cascade is a series of reactions. Nucleic acid strand transduction is a reaction where one input nucleic acid strand is replaced with another output nucleic acid strand. Thresholded transduction is transduction predicated on a threshold quantity of the input nucleic acid strand. A reaction such as amplification or transduction is armored if it is not able to occur without appropriate initiation. The designs disclosed herein enable the construction of effective nucleic acid reaction cascades including but not limited to (1) armored amplification (FIGS. 1-3), (2) armored transduction plus amplification (FIGS. 4-6), (3) thresholded transduction plus amplification (FIGS. 7), and (4) armored/thresholded transduction plus reactivation of amplification (FIG. 8). Furthermore, the inherent thermodynamic properties of competitive hybridization and strand displacement also enable sensitive discrimination of single-nucleotide mismatch¹³⁻¹⁵ during target recognition, which directly facilitates mutant rule-in and rule-out for multiplexed molecular diagnostic applications (FIG. 9).

It is to be understood that the molecular mechanisms disclosed herein (including armoring, de-armoring, thresholding, de-thresholding, deactivating, reactivating) are not limited to the example designs of nucleic acid modules described herein. For example, the basic nucleic acid components of the disclosed reaction protocols (e.g., the armor strand, threshold complex, deactivation strand, etc.) can be designed and optimized according to requirements of the intended assay with flexible choice of the hybridization site, length, stability, use of mismatched bases, toehold, and variants or modifications of nucleic acid sequences, for example. In some embodiments, for example, the nucleic acid modules may be designed to leverage DNA hairpins¹⁶ instead of the basic duplex-form constructs. Some embodiments may leverage the use of nucleic acid analogues such as PNA and LNA¹⁷⁻¹⁹ in place of or in combination with DNA and/or RNA to achieve desired thermodynamic properties of the molecular mechanism.

It is also to be understood that the applications of the disclosed molecular mechanisms are not limited to specific methods or implementations of molecular detection, transduction, amplification, and/or readout systems unless otherwise specified. For example, the disclosed molecular mechanisms can be utilized to enhance the detection of nucleic acid targets including but are not limited to DNA, dsDNA, ssDNA, RNA, mRNA, microRNA, tRNA, rRNA, sgRNA, siRNA, and analogs of the foregoing such as PNA, LNA, TNA, HNA, GNA, and the like.

In some embodiments, the disclosed molecular mechanisms may be leveraged to improve the specificity of various isothermal amplification techniques other than the conventional LAMP and/or RT-LAMP methods, for example, including but not limited to variations of LAMP-like amplification techniques based on dual-priming, swarm priming, stem priming, hairpin primers, etcetera.²⁰⁻²⁵ Some of the underlying mechanisms proposed herein (e.g., armoring and un-armoring) may also be strategically utilized to enhance the performance of conventional thermocycling-based amplification methods such as PCR, RT-PCR, qPCR, and RT-qPCR.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification.

FIGS. 1A-1C illustrates an exemplary scheme of armored primer and amplification protocol.

FIG. 2 illustrates the components of an exemplary scheme of armored LAMP protocol.

FIGS. 3A and 3B illustrates the reaction pathway of an exemplary scheme of armored LAMP protocol.

FIG. 4 illustrates the components of an exemplary scheme of armored transduction protocol.

FIG. 5 illustrates the reaction pathway of an exemplary scheme of armored transduction protocol.

FIGS. 6A and 6B illustrates a universal LAMP protocol initiated by an un-armored transduction strand.

FIGS. 7A-7C illustrate an exemplary scheme of thresholded transduction and amplification protocol.

FIGS. 8A-8C illustrate an exemplary scheme of reactivation of deactivated primer and amplification.

FIGS. 9A and 9B illustrate a mechanism of ultra-specific target discrimination by use of armored primers.

### DETAILED DESCRIPTION

### OVERVIEW

The present disclosure describes nucleic-acid-based molecular mechanisms that may be utilized to address the non-specific amplification (false positive) limitations associated with conventional isothermal amplification protocols such as LAMP and RT-LAMP. The underlying principles can also be custom-programmed and broadly applied to enhance the specificity and signal-to-noise ratio of other molecular detection, transduction, amplification, and readout protocols.

### Armoring/De-armoring

In some embodiments, armoring can be achieved by the introduction of a short single-stranded nucleic acid “armor strand” that contains a subsequence of sufficient length complementary to the 3′ segment of a primer used in a nucleic acid detection, transduction, or amplification protocol. The 3′ end of the armor strand is designed to deter polymerase extension (for example, by appending a short overhang “toehold”, incorporating a modification such as 3′ inverted dT, 3′ ddC, 3′ C3 spacer, 3′ amino, or 3′ phosphorylation that blocks extension by DNA polymerase, incorporating nonstandard nucleic acid bases, or G quadruplex).

During the reaction of an assay, the armor strand is present in the solution at moderately high concentration such that its competitive binding to the primer enforces a higher energy threshold for the occurrence of complete primer binding to the priming site on the target sequence, thereby increasing the stringency for the onset of target-specific amplification. In some embodiments, a “moderately high concentration” for the armor strand can be a concentration that is 0.05×, 0.1×, 0.2×, 0.3×, 0.4×, 0.5×, 0.6×, 0.7×, 0.8×, 0.9×, 1.0×, 1.1×, 1.2×, 1.3×, 1.4×, 1.5×, 1.6×, 1.7×, 1.8×, 1.9×, or 2.0× the concentration of the corresponding primer to which it binds, or can be within a range using any combination of the foregoing as endpoints.

With appropriately adjusted concentration and thermodynamic parameters of the armor strand, the hybridization between the armor strand and the primer can help to block or at least transiently sequester the 3′ end of the primer to effectively inhibit spurious primer-primer and primer-template interactions to reduce false positives. Further, once a primer is armored (i.e., bound with its armor strand), it must undergo an initial partial binding to the target priming site then followed by branch migration and strand displacement to release the initially bound armor strand (i.e., de-armoring) before the primer can fully hybridize to its target sequence and initialize polymerase extension.

In some embodiments, the binding site and sequences of the primer and armor strand can be adjusted and optimized to discriminate single nucleotide mutations on the target DNA or RNA owing to the high specificity driven by thermodynamic properties of nucleic acid strand displacement reactions. The extent of armoring can be controlled by adjusting the initial concentration of the armor strand in solution. In some embodiments, de-armoring refers to the reduction in armor strand concentration or the absence of armor strand in reaction. Furthermore, depending on the implementation and performance requirements of the assay, some embodiments can leverage one or more armored primers in a one-pot reaction.

### Threshold Complexes

In some embodiments, thresholding can be achieved by the introduction of a pre-annealed oligo dimer termed a “threshold complex”, which is designed to consume an input strand up to a pre-determined threshold level before the input strand can effectively trigger any downstream reactions. In some embodiments, the input to the threshold complex can be a target-transduced strand (released from an upstream transduction protocol) that functions as a primer for a downstream amplification protocol. The thresholding mechanism is designed to suppress potential background leakage (i.e., unintended triggering of downstream reactions due to spurious exposure of binding domains from upstream reaction components) that may occur in cascaded reaction pathways of an assay, caused by factors such as mis-formed or non-annealed nucleic acid complexes.

The input strand (which functions as the “primer” for a downstream amplification protocol) consumed by thresholding is rendered in the form of an inert waste species that in some embodiments can be simply a blunt-ended duplex. Reaction between the primer and the threshold complex also results in the release of a single-stranded nucleic acid oligo called “moderator strand” from the threshold complex. Because the moderator strand contains a subsequence identical to the 5′ segment of the primer sequence, the moderator strand (once freed from the threshold complex) can compete with the primer in binding with the downstream amplification priming site. Such a competitive binding may also be designed to limit initiation of false amplification by inclusion of a 3′ modification (e.g., short overhang toehold, 3′ inverted dT, 3′ ddC, 3′ C3 spacer, 3′ amino, 3′ phosphorylation, nonstandard nucleic acid bases, or G quadruplex) on the moderator strand that prevents polymerase extension. These molecular mechanisms decrease leakage and increase specificity simultaneously.

The extent of thresholding can be controlled by adjusting the initial concentration of the threshold complex in solution. In some embodiments, de-thresholding refers to the reduction in threshold complex concentration or the absence of threshold complex in reaction. Furthermore, depending on the implementation and performance requirements of the assay, some embodiments can leverage threshold complexes for one or more primers or transduced strands in a one-pot reaction.

In some embodiments, the thresholding mechanism may be combined with the armoring mechanism in a single assay and/or reaction mixture.

### Deactivated/Reactivated Primers

In some embodiments, a primer for a downstream amplification protocol is initially deactivated by annealing to a deactivation strand to form a “deactivated primer” dimer including a hybridized domain of sufficient length that it fully sequesters the primer at its 3′ end. This pre-annealed dimer has an extended overhang at the 3′ end of the deactivation strand. In some embodiments, the transduced strand released from an upstream detection and transduction protocol are designed to function as a “reactivation strand”, which recognizes the exposed overhang of the deactivated primer and triggers strand displacement to release the initially bound primer from the deactivated primer dimer. Because the reactivation strand contains a subsequence identical to the 3′ segment of the primer sequence, the reactivation strand (once released from the upstream detection or transduction protocol) can compete with the primer in binding with the downstream amplification priming site. Such a competitive binding may also be designed to limit initiation of false amplification by way of a 3′ modification (e.g., short overhang toehold, 3′ inverted dT, 3′ ddC, 3′ C3 spacer, 3′ amino, or 3′ phosphorylation, nonstandard nucleic acid bases, or G quadruplex) on the reactivation strand that prevents polymerase extension.

In some embodiments, the deactivated primer mechanism can be used in tandem with the armoring and/or thresholding mechanism to offer another layer of protection against the generation of false positive amplicons in molecular assays. Furthermore, depending on the implementation and performance requirements of the assay, some embodiments can leverage one or more deactivated primers in a one-pot reaction.

### EXAMPLE EMBODIMENTS

### Armoring/De-armoring

In the illustrations and accompanying descriptions disclosed below, an asterisk (*) denotes sequence reverse complementarity. For example, the complementary sequence of oligo a is denoted by a*.

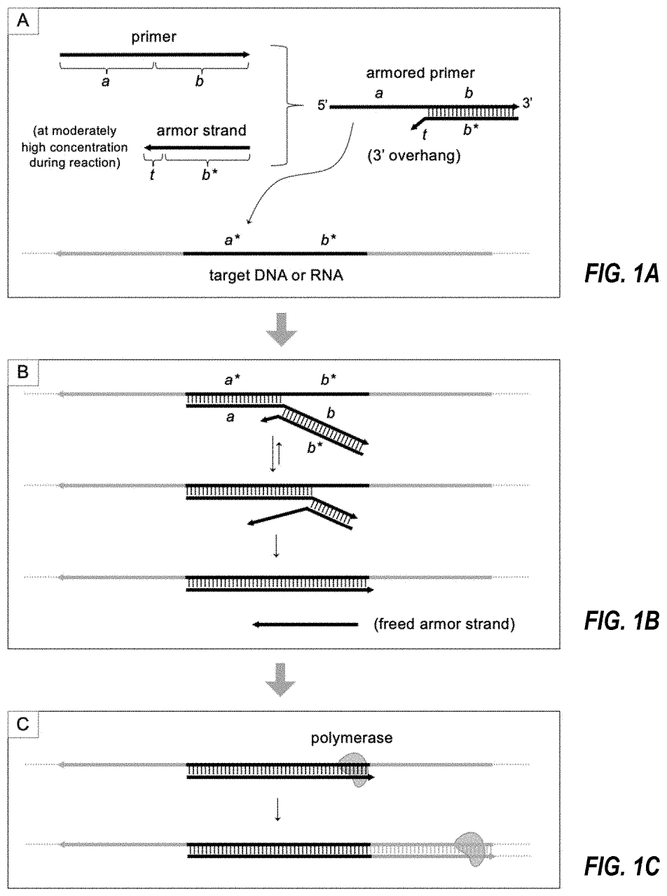

In FIGS. 1A-1C, an example scheme of the armored primer and amplification protocol is illustrated. FIG. 1A illustrates the sequence composition of the primer and the armor strand. The primer comprises two subsequences (also conceptually viewed as functional “domains”) a and b listed from the 5′ to 3′ orientation. The armor strand contains the domain b* that is reverse complementary to the domain b on the primer. The armor strand also contains a simple 3′ modification (as indicated by the letter t in the drawings) such as a short overhang toehold or an inverted dT or other 3′ modification disclosed herein to prevent polymerase extension. The armor strand may also contain additional subsequences in its prefix or suffix, but these are not illustrated in this Figure for clarity.

Some of the armor strands are initially unbound and free in solution at moderately high concentration. Hybridization between the primer and a free armor strand forms an armored primer that leaves the 5′ subsequence (i.e., domain a) of the primer exposed and free to bind with the target DNA or RNA via the corresponding subsequence (i.e., domain a*) of the priming site. FIG. 1B illustrates that the armor strand and the target sequence compete to bind with the primer in the solution. Once an armored primer binds to the target sequence, the process of branch migration proceeds and strand displacement eventually frees the single-stranded armor strand back into the solution. This process is thermodynamically highly specific and is favorably driven forward by the formation of a perfect match between the primer and the target sequences. As shown in FIG. 1C, the polymerase starts DNA synthesis from the 3′ end of the now fully hybridized primer on the target to initiate the downstream amplification protocol.

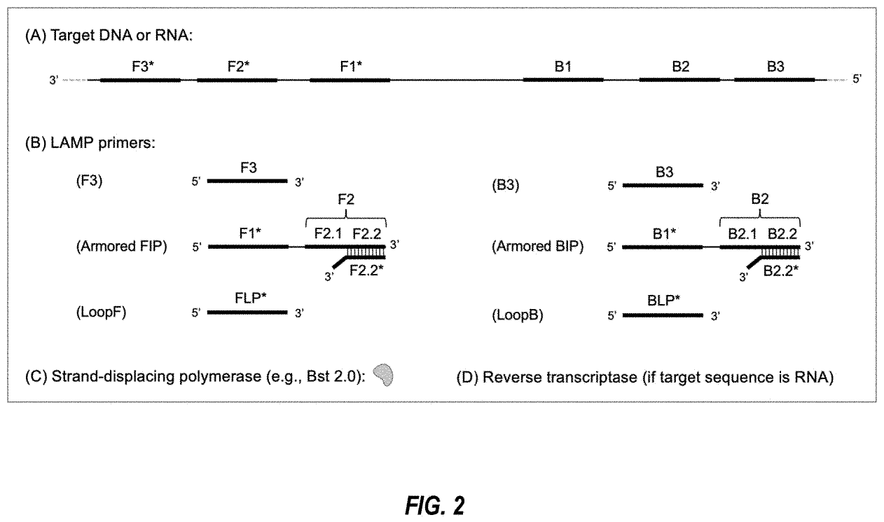

In FIG. 2, the reaction components of an exemplary scheme of armored LAMP protocol are illustrated. For brevity, common reagents such as reaction buffer and nuclease-free water are not depicted in the drawing. Component (A) illustrates the sequence composition of a target DNA or RNA template for the LAMP reaction. The subsequences F3*, F2*, F1*, B1, B2, B3 listed from the 3′ to 5′ orientation on the target sequence correspond to the six primer binding sites described in conventional LAMP protocols.

Components (B) are the set of LAMP primers designed for isothermal amplification of the target. Specifically, the set comprises the F3 primer, the B3 primer, the LoopF primer, the LoopB primer, with the armored FIP primer and the armored BIP primer. The armored FIP primer is formed by a primer nucleic acid strand consisting of subsequences F1* and F2 (listed from the 5′ to 3′ orientation) and an armor strand that hybridizes to the F2.2 subdomain of the F2 subsequence on the primer nucleic acid strand. The armor strand contains a simple 3′ modification such as a short overhang toehold, an inverted dT, or other 3′ modification as disclosed herein to prevent polymerase extension.

Similarly, the armored BIP primer is formed by a primer nucleic acid strand consisting of subsequences B1* and B2 (listed from the 5′ to 3′ orientation) and an armor strand that hybridizes to the B2.2 subdomain of the B2 subsequence on the primer nucleic acid strand. The armor strand contains a simple 3′ modification such as a short overhang toehold, an inverted dT, or other 3′ modification disclosed herein to prevent polymerase extension. In some embodiments, other essential primers (e.g., F3 primer and/or B3 primer) from the LAMP primer set can be armored in a similar fashion. Component C indicates that the reaction contains a strand-displacing polymerase such as Bst 2.0. Component D indicates that the reaction may also contain a reverse transcriptase if the amplification target is RNA sequence.

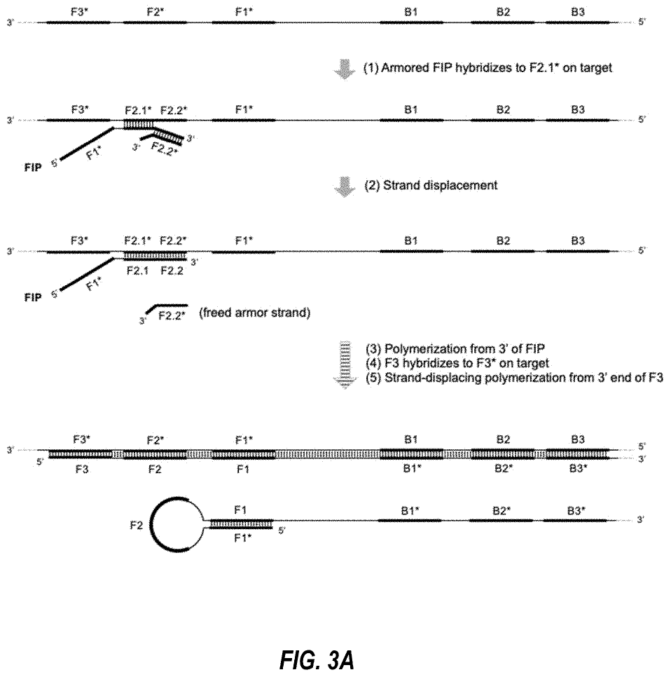

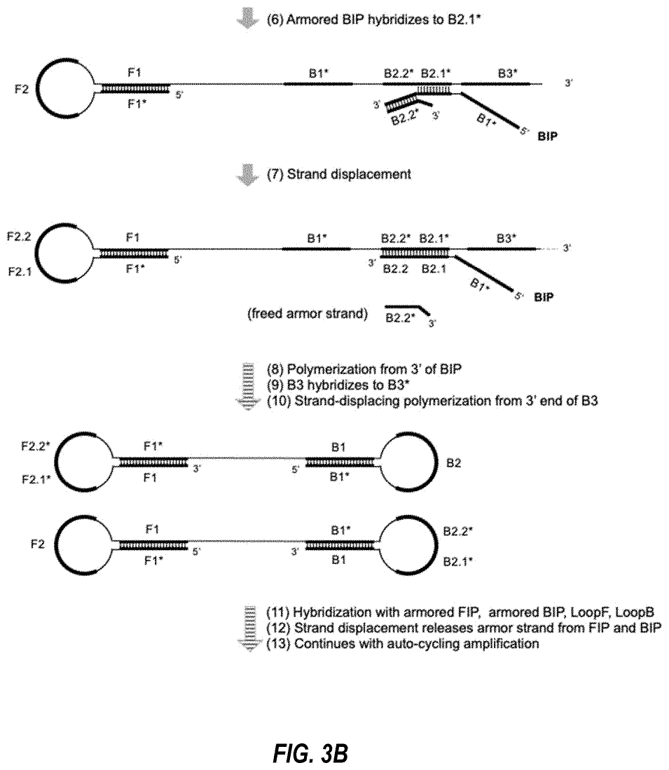

In FIGS. 3A and 3B, the reaction pathway of an exemplary scheme of armored LAMP protocol is illustrated. Step (1) illustrates that the armored FIP primer hybridizes to the F2.1* subsequence of the F2* priming site on the target template. Step (2) indicates the process of branch migration and strand displacement resulting in full hybridization between the F2 subsequence on the FIP primer and the F2* subsequence on the target template. At the same time, the armor strand is released from the armored FIP primer and become free to armor another unbound FIP primer in the solution.

Steps (3-10) illustrate a series of primer extension and strand-displacing polymerization reactions of LAMP that leads to the formation of the basic dumbbell DNA structure that serves as the starting material for self-primed auto-cycling amplification reaction of LAMP. Specifically, Steps (3-5) indicate the processes including the primer extension from the 3′ end of the fully hybridized FIP primer on target, followed by binding of the F3 primer on the target that primes strand-displacing polymerization to release the newly synthesized strand primed by FIP. Step (6) illustrates that the armored BIP primer hybridizes to the B2.1* subsequence of the B2* priming site on the newly formed DNA structure from the previous step. Step (7) indicates the process of branch migration and strand displacement resulting in full hybridization between the B2 subsequence of the BIP primer and the B2* subsequence of the newly formed DNA structure. At the same time, the armor strand is released from the armored BIP primer and become free to armor another unbound BIP primer in the solution. Steps (8-10) indicates the processes including the primer extension from the 3′ end of the fully hybridized BIP primer, followed by binding of the B3 primer to the priming site B3*, which primes strand-displacing polymerization to release the newly synthesized strand primed by BIP and result in the formation of the basic dumbbell DNA structures.

Steps (11-13) indicate the initialization of the LAMP auto-cycling amplification. These downstream reactions involve the participation of the armored FIP primer, the armored BIP primer, and the loop primers LoopF and LoopB. Note that the effect of armoring on FIP and BIP is also present in these reactions, which helps to maintain specificity during exponential amplification. For brevity, detailed illustrations for the hybridization, strand displacement, and strand-displacing polymerization reactions involved in these processes, which are known in the art, are not shown.

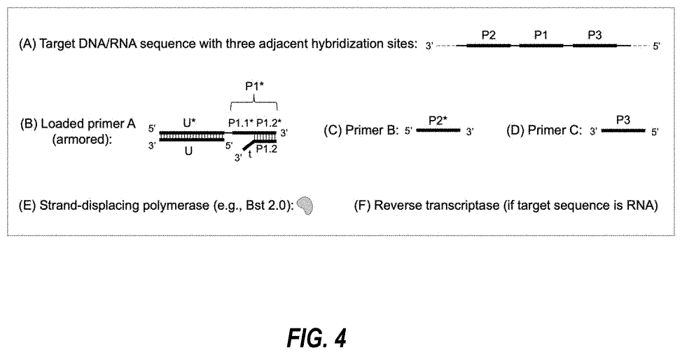

In FIG. 4, the reaction components of an exemplary scheme of an armored transduction protocol are illustrated. For the exemplary implementation demonstrated herein, the underlying principle of nucleic acid target transduction is similar to that described in U.S. patent application Ser. No. 17/749,858, which is incorporated herein by reference in its entirety, with the exception that the armoring mechanism is applied herein to one or more of the primers used during transduction. Briefly, the transduction protocol can be used to detect the presence of any target DNA or RNA sequence into the release of one or more copies of a universal single-stranded nucleic acid oligo, which functions as one of the essential primers capable of triggering a downstream universal LAMP protocol that is predesigned, independent of the target sequence, and highly optimized in performance.

Component (A) illustrates that the target DNA or RNA sequence contain three adjacent hybridization sites recognized by the transduction protocol, including P2, P1, and P3 (listed from the 3′ to 5′ orientation). Component (B) illustrates an armored scheme for the loaded primer A, consisting of a primer with subsequence U* and P1* (listed from the 3′ to 5′ orientation) hybridized to a transduction strand U on the 5′ end and an armor strand on the 3′ end, respectively. The armor strand contains a simple 3′ modification (as indicated by the letter tin the drawing) such as a short overhang toehold or an inverted dT to prevent polymerase extension. The armor strand is initially unbound and free in solution at moderately high concentration. Hybridization between the loaded primer A and the armor strand forms an armored primer that leaves the P1.1* subsequence of the primer exposed and free to bind with the target DNA or RNA.

Component (C) illustrates a primer B consisting of nucleic acid sequence P2*. Component (D) illustrates a primer C consisting of nucleic acid sequence P3. In some embodiments, the primer B and/or primer C may be armored in a similar fashion described above. Component (E) indicates that the reaction contains a strand-displacing polymerase such as Bst 2.0. Component (F) indicates that the reaction may also contain a reverse transcriptase if the transduction target is RNA sequence.

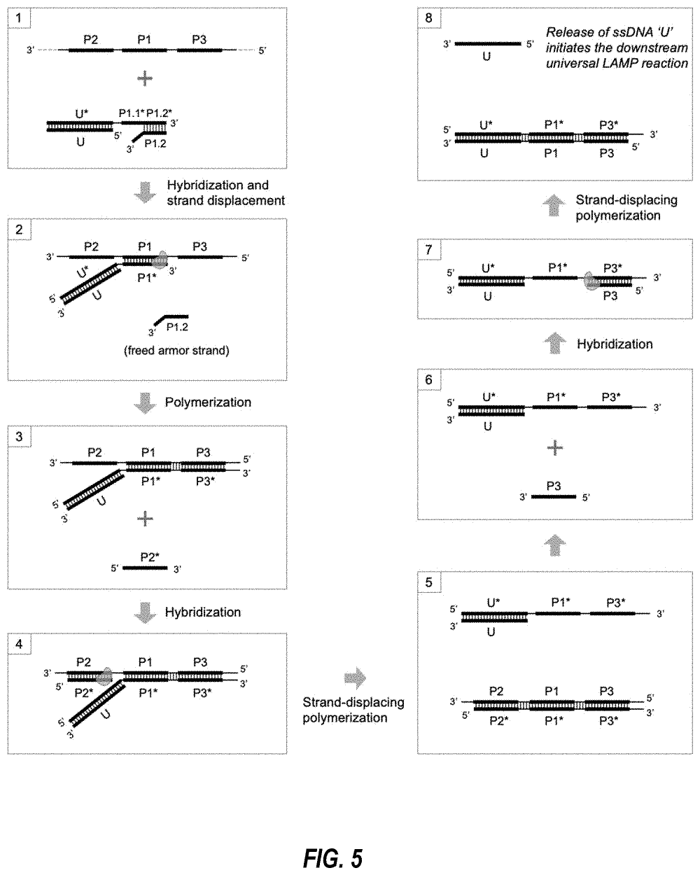

In FIG. 5, the reaction pathway of an exemplary scheme of armored transduction protocol is illustrated. For the exemplary implementation demonstrated herein, the underlying transduction reaction pathway is similar to that described in U.S. patent application Ser. No. 17/749,858, which is incorporated herein by reference in its entirety, with the exception that the armoring mechanism is applied herein to the loaded primer A such that its full hybridization to the target priming site must undergo an initial partial binding followed by branch migration and strand displacement to release the armor strand before primer extension can proceed from the 3′ end of the loaded primer A.

In FIG. 5, Step (1) illustrates that the armored loaded primer A can hybridize to a subsequence of the P1 priming site on the target via the exposed P1.1* subsequence of the armored loaded primer, with subsequent strand displacement following hybridization of the P.1.2* subsequence to the target. Step (2) illustrates that the strand displacement leads to the release of armor strand from the armored loaded primer, which is then able to initialize polymerization from the 3′ of P1*. Step (3) illustrates that the primer B binds to the priming site P2 on the target. Step (4) illustrates the strand-displacing polymerization reaction initialized by the primer B. Step (5) illustrates the resultant nucleic acid species produced from the strand-displacing polymerization reaction. Step (6) illustrates that the primer C binds to the priming site P3* on the resultant nucleic acid species. Step (7) illustrates the strand-displacing polymerization reaction initialized by the primer C. Step (8) illustrates the resultant nucleic species produced from the strand-displacing polymerization reaction, including the release of the transduction strand U that is designed to trigger a downstream universal LAMP protocol.

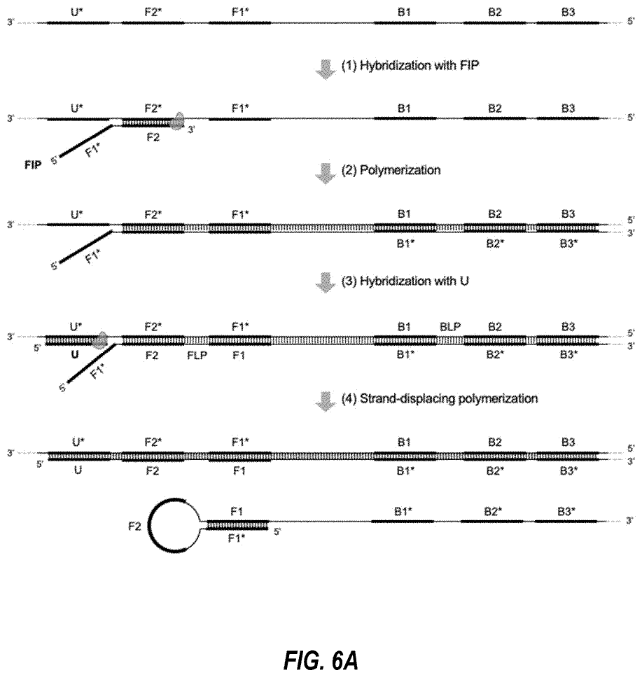

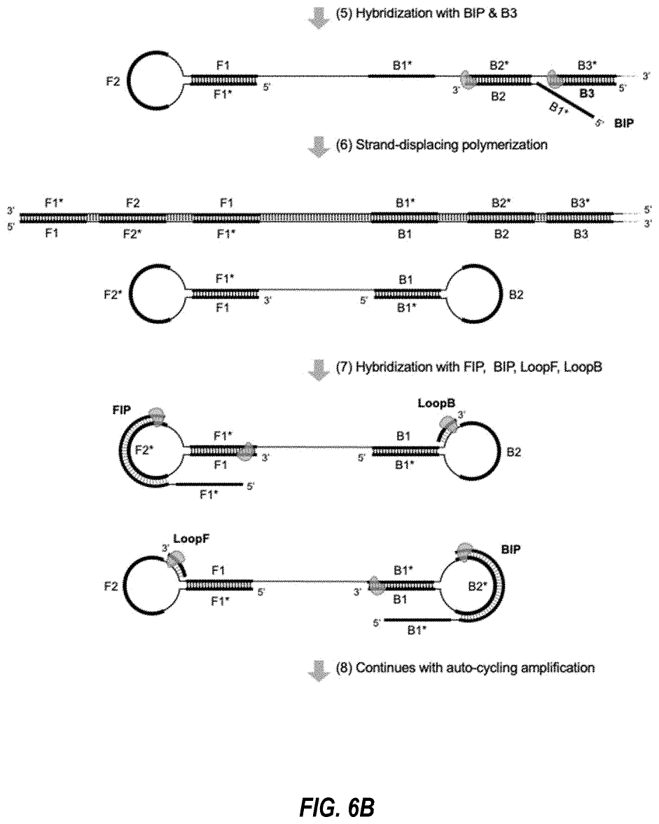

In FIGS. 6A and 6B, the initialization of a universal LAMP protocol by an un-armored transduction strand is illustrated. The exemplary reaction pathway illustrated herein is similar to the conventional LAMP protocol with the exception that the transduction strand U released from the upstream transduction protocol replaces the F3 primer in the present implementation. In some embodiments, the transduction strand U may function as one of the other essential primers in conventional LAMP protocol. Step (1) illustrates the hybridization of FIP to its priming site on a universal LAMP template. Step (2) illustrates the polymerization initiated from the 3′ end of FIP. Step (3) illustrates the hybridization of U to its priming site on the universal LAMP template. Step (4) illustrates the strand-displacing polymerization initiated from the 3′ end of U, releasing the FIP-primed newly synthesized strand. Step (5) illustrates that the BIP primer and the B3 primer hybridizes to their respective priming sites on the newly synthesized strand released from the previous step. Step (6) illustrates the polymerization initiated from the 3′ end of BIP and strand-displacing polymerization initiated from the 3′ end of B3, resulting in the formation of the basic dumbbell DNA structure of LAMP. Steps (7-8) indicate the initialization of the LAMP auto-cycling amplification, facilitated by self-primed displacement synthesis using FIP, BIP, LoopF, and LoopB primers.

### Threshold Complexes

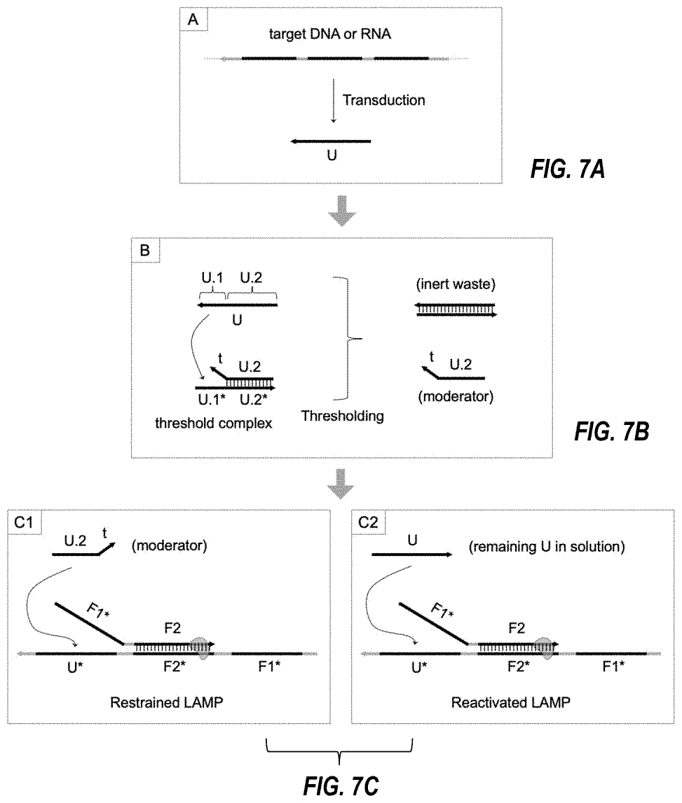

In FIGS. 7A-7C, an exemplary scheme of thresholded transduction and amplification protocol is illustrated. Step (A) illustrates that the presence of a target DNA or RNA sequence is detected by a transduction protocol (such as any of those disclosed herein) and results in the release of a transduced strand U. Step (B) illustrates the thresholding mechanism that converts a predetermined amount of U into inert DNA species. Specifically, the transduced strand U released from the upstream reaction contains functional domains U.1 and U.2 listed from the 3′ to 5′ orientation. Also present in the reaction mixture is a pre-annealed DNA dimer called the “threshold complex”, which is formed by a “moderator strand” hybridized to the U.2* domain on a nucleic acid strand consisting of subsequences U.1* and U.2* listed from the 5′ to 3′ end. The moderator strand contains the domain U.2 and a simple 3′ modification (as indicated by the letter t in the drawings) such as a short overhang toehold, an inverted dT, or other 3′ modification as disclosed herein to prevent polymerase extension.

During thresholding, the transduced strand U binds to the exposed U.1* domain on the threshold complex, and via strand displacement, releases the moderator strand from the threshold complex and at the same time, consumes the single-stranded transduced U into the formation of a blunt-ended duplex that is functionally inert and can be viewed as a waste species in the solution. In some embodiments, the threshold level can be simply adjusted by varying the concentration of the threshold complex.

Step (C1) illustrates that the moderator strand released from the thresholding reaction can compete with the transduced strand U (which functions as an essential primer for a downstream amplification protocol) in binding with the target priming site. The competitive binding of moderator strand, however, does not trigger false amplification owing to the presence of its 3′ modification that deters polymerase extension.

Step (C2) illustrates that after thresholding, the remaining transduced strands U from the solution can fully hybridize to the target priming site and trigger the downstream amplification. Such an initialization of the amplification protocol is only effective if the total amount of U released from the upstream transduction protocol sufficiently exceeds the threshold level set by the thresholding reaction.

### Deactivated/Reactivated Primers

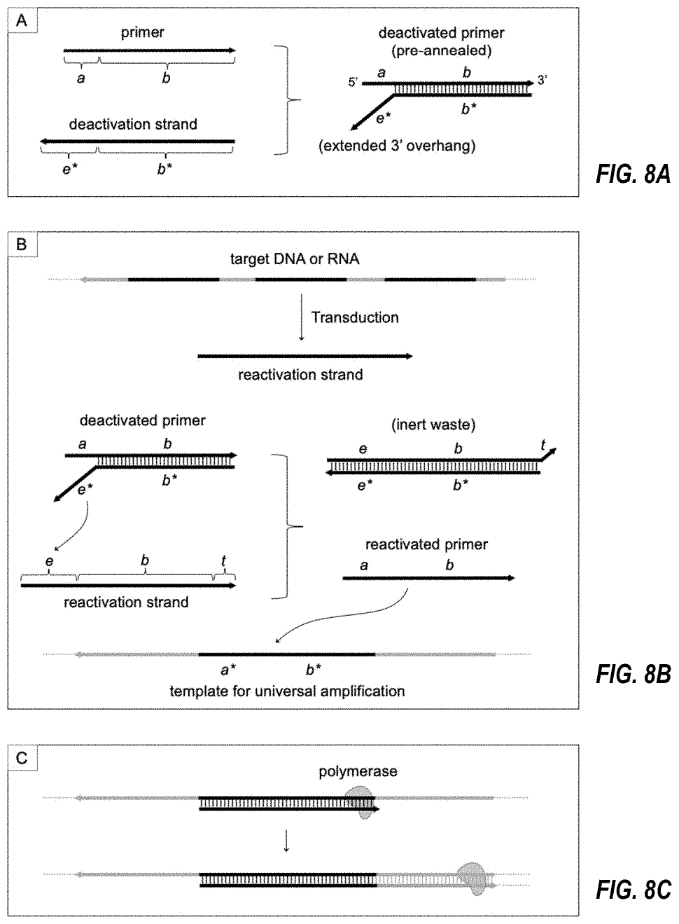

In FIGS. 8A-8C, an exemplary scheme for reactivation of deactivated primer and amplification is illustrated. Step (A) illustrates the formation of a deactivated primer through annealing of the primer (containing subsequences a and b listed from the 5′ to 3′ orientation) to a deactivation strand (containing subsequences e* and b* listed from the 3′ to 5′ orientation). The annealed deactivated primer contains a fully hybridized b/b* domain while the subsequences a and e* both remain unhybridized.

Step (B) illustrates that the presence of a target DNA or RNA sequence is detected by a transduction protocol (such as disclosed herein) and results in the release of a transduced strand which functions as a “reactivation strand” in the following reactions. For example, the reactivation strand consists of subsequences e, b, and t listed from the 5′ to 3′ orientation, and through the binding of subsequence e of the reactivation strand to the extended 3′ overhang sequence e* of the deactivated primer, strand displacement reaction can proceed and lead to the release of the primer from the deactivated primer dimer. This released primer is now considered reactivated and is free to hybridize to its target priming site (i.e., a* b*) on a downstream amplification template, which, in some embodiments, can be a universal template for a highly optimized amplification protocol. The reactivation reaction also generates an inert duplex that is functionally considered a waste species. Step (C) illustrates that the reactivated primer fully hybridizes to the target template to initiate the downstream amplification protocol.

### Specific Nucleic Acid Discrimination

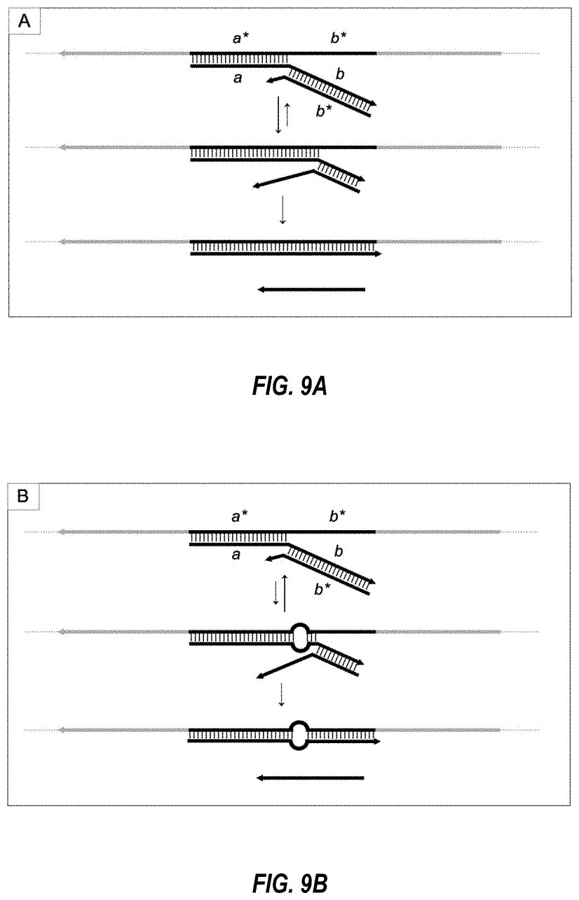

FIGS. 9A and 9B illustrate a mechanism of specific nucleic acid target discrimination by use of an armored primer. Specifically, thermodynamic properties of competitive hybridization and strand displacement can be utilized to discriminate single-nucleotide mismatch between the primer and target sequences.

FIG. 9A illustrates the case of perfect match between the primer and the target sequences. Under such a condition, fast forward strand displacement is favored and quickly results in the release of the armor strand from the armored primer. In contrast, FIG. 9B illustrates that strand displacement with one or more nucleotide mismatches is thermodynamically less favorable and thus cannot proceed efficiently in the forward direction to lead to full hybridization between the primer and the target to facilitate amplification. The location of the mismatched nucleotide(s) can vary in different embodiments and may be strategically selected to maximize the signal-to-noise ratio for single-nucleotide discrimination.

### ADDITIONAL TERMS & DEFINITIONS

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, numbers expressing quantities, constituents, distances, or other measurements used in the specification and claims may optionally be modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, amplification reaction mixture components not specifically disclosed and/or not necessarily required to carry out the disclosed methods may be completely omitted or essentially omitted from the disclosed embodiments.

An embodiment that “essentially omits” or is “essentially free of” a component may include trace amounts and/or non-functional amounts of the component. For example, an “essentially omitted” component may be included in an amount no more than 2.5%, no more than 1%, no more than 0.1%, or no more than 0.01% by total weight or total volume of the reaction mixture. This is likewise applicable to other negative modifier phrases such as “essentially omits,” “essentially without,” similar phrases using “substantially” or other synonyms of “essentially.”

Any headings and subheadings used herein are for organizational purposes only and are not meant to be used to limit the scope of the description or the claims. References provided herein are incorporated herein by reference.

It will also be noted that, as used in this specification and the appended claims, the singular forms “a,” “an” and “the” do not exclude plural referents unless the context clearly dictates otherwise.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### REFERENCES

(1) Meagher, R. J.; Priye, A.; Light, Y. K.; Huang, C.; Wang, E. Impact of Primer Dimers and Self-Amplifying Hairpins on Reverse Transcription Loop-Mediated Isothermal Amplification Detection of Viral RNA. Analyst 2018, 143, 1924-1933.
(2) Moehling, T. J.; Choi, G.; Dugan, L. C.; Salit, M.; Meagher, R. J. LAMP Diagnostics at the Point-of-Care: Emerging Trends and Perspectives for the Developer Community. Expert Rev. Mol. Diagn. 2021, 21, 43-61.
(3) Özay, B.; McCalla, S. E. A Review of Reaction Enhancement Strategies for Isothermal Nucleic Acid Amplification Reactions. Sensors and Actuators Reports 2021, 3, 100033.
(4) Ball, C. S.; Light, Y. K.; Koh, C.-Y.; Wheeler, S. S.; Coffey, L. L.; Meagher, R. J. Quenching of Unincorporated Amplification Signal Reporters in Reverse-Transcription Loop-Mediated Isothermal Amplification Enabling Bright, Single-Step, Closed-Tube, and Multiplexed Detection of RNA Viruses. Anal. Chem. 2016, 88, 3562-3568.
(5) Jiang, Y. S.; Bhadra, S.; Li, B.; Wu, Y. R.; Milligan, J. N.; Ellington, A. D. Robust Strand Exchange Reactions for the Sequence-Specific, Real-Time Detection of Nucleic Acid Amplicons. Anal. Chem. 2015, 87, 3314-3320.
(6) Tanner, N. A.; Zhang, Y.; Evans, T. C. Simultaneous Multiple Target Detection in Real-Time Loop-Mediated Isothermal Amplification. Biotechniques 2012, 53, 81-89.
(7) Becherer, L.; Bakheit, M.; Frischmann, S.; Stinco, S.; Borst, N.; Zengerle, R.; von Steffen, F. Simplified Real-Time Multiplex Detection of Loop-Mediated Isothermal Amplification Using Novel Mediator Displacement Probes with Universal Reporters. Anal. Chem. 2018, 90, 4741-4748.
(8) R. Kubota; A. M. Alvarez; W.W. Su; D. M. Jenkins. FRET-Based Assimilating Probe for Sequence-Specific Real-Time Monitoring of Loop-Mediated Isothermal Amplification (LAMP). Biol. Eng. Trans. 2011, 4, 81-100.
(9) Seelig, G.; Soloveichik, D.; Zhang, D. Y.; Winfree, E. Enzyme-Free Nucleic Acid Logic Circuits. Science (80-.). 2006, 314, 1585-1588.
(10) Zhang, D. Y.; Turberfield, A. J.; Yurke, B.; Winfree, E. Engineering Entropy-Driven Reactions and Networks Catalyzed by DNA. Science (80-.). 2007, 318, 1121-1125.
(11) Yin, P.; Choi, H. M. T.; Calvert, C. R.; Pierce, N. A. Programming Biomolecular Self-Assembly Pathways. Nature 2008, 451, 318-322.
(12) Zhang, D. Y.; Seelig, G. Dynamic DNA Nanotechnology Using Strand-Displacement Reactions. Nat. Chem. 2011, 3, 103-113.
(13) Zhang, D. Y.; Chen, S. X.; Yin, P. Optimizing the Specificity of Nucleic Acid Hybridization. Nat. Chem. 2012, 4, 208-214.
(14) Jiang, Y. S.; Bhadra, S.; Li, B.; Ellington, A. D. Mismatches Improve the Performance of Strand-Displacement Nucleic Acid Circuits. Angew. Chemie Int. Ed. 2014, 53, 1845-1848.
(15) Tang, W.; Zhong, W.; Tan, Y.; Wang, G. A.; Li, F.; Liu, Y. DNA Strand Displacement Reaction: A Powerful Tool for Discriminating Single Nucleotide Variants. Top. Curr. Chem. 2020, 378, 10.
(16) Green, S. J.; Lubrich, D.; Turberfield, A. J. DNA Hairpins: Fuel for Autonomous DNA Devices. Biophys. J. 2006, 91, 2966-2975.
(17) Kundu, N.; Young, B. E.; Sczepanski, J. T. Kinetics of Heterochiral Strand Displacement from PNA-DNA Heteroduplexes. Nucleic Acids Res. 2021, 49, 6114-6127.
(18) Reid, M. S.; Paliwoda, R. E.; Zhang, H.; Le, X. C. Reduction of Background Generated from Template-Template Hybridizations in the Exponential Amplification Reaction. Anal. Chem. 2018, 90, 11033-11039.
(19) Itonaga, M.; Matsuzaki, I.; Warigaya, K.; Tamura, T.; Shimizu, Y.; Fujimoto, M.; Kojima, F.; Ichinose, M.; Murata, S. Novel Methodology for Rapid Detection of KRAS Mutation Using PNA-LNA Mediated Loop-Mediated Isothermal Amplification. PLoS One 2016, 11, e0151654.
(20) Zhao, Y.; Chen, F.; Li, Q.; Wang, L.; Fan, C. Isothermal Amplification of Nucleic Acids. Chem. Rev. 2015, 115, 12491-12545.
(21) Gill, P.; Ghaemi, A. Nucleic Acid Isothermal Amplification Technologies—A Review. Nucleosides, Nucleotides and Nucleic Acids 2008, 27, 224-243.
(22) Ding, X.; Xu, Z.; Yin, K.; Sfeir, M.; Liu, C. Dual-Priming Isothermal Amplification (DAMP) for Highly Sensitive and Specific Molecular Detection with Ultralow Nonspecific Signals. Anal. Chem. 2019, 91, 12852-12858.
(23) Ding, X.; Wang, G.; Mu, Y. Single Enzyme-Based Stem-Loop and Linear Primers Co-Mediated Exponential Amplification of Short Gene Sequences. Anal. Chim. Acta 2019, 1081, 193-199.
(24) Yin, Y.; Wu, Z.; Li, G.; Huang, J.; Guo, Q.; Meng, X. A DNA Molecular Diagnostic Technology with LAMP-like Sensitivity Based on One Pair of Hairpin Primers-Mediated Isothermal Polymerization Amplification. Anal. Chim. Acta 2020, 1134, 144-149.
(25) Martineau, R. L.; Murray, S. A.; Ci, S.; Gao, W.; Chao, S.; Meldrum, D. R. Improved Performance of Loop-Mediated Isothermal Amplification Assays via Swarm Priming. Anal. Chem. 2016, 89, 625-632.

1. A composition for isothermal amplification of a target nucleic acid, the composition comprising:
a first primer that includes a 5′ section and a 3′ section; and
an armor strand that includes a complementary section and a 3′ modification, the complementary section being complementary to the 3′ section of the first primer such that when the armor strand hybridizes to the first primer, the 3′ section of the first primer becomes sequestered while the 5′ section of the first primer is free to hybridize to a first target location of a target nucleic acid; and
at least a second primer associated with a second target location of the target nucleic acid to enable, with the first primer, amplification of the target nucleic acid,
wherein during the amplification, the 3′ modification of the armor strand prevents polymerase extension of the armor strand.

2. The composition of claim 1, wherein the 3′ modification is a 3′ overhang, 3′ inverted dT, 3′ ddC, 3′ C3 spacer, 3′ amino, 3′ phosphorylation that blocks extension by polymerase, one or more nonstandard nucleic acid bases, or G quadruplex.

3. The composition of claim 1, further comprising additional primers that form, with the first and second primer, a full set of primers for an isothermal amplification reaction.

4. The composition of claim 1, wherein the first and second primer, optionally with one or more additional primers, are configured to enable a transduction reaction that generates a transduction product when the target nucleic acid is present, the transduction product functioning to modulate a downstream amplification reaction.

5. The composition of claim 4, wherein the downstream amplification reaction comprises a LAMP or LAMP-like isothermal amplification reaction, wherein the composition further comprises an incomplete set of primers for the LAMP or LAMP-like reaction, the transduction product functioning as a required primer to enable the downstream amplification reaction.

6. The composition of claim 1, further comprising a DNA polymerase.

7. The composition of claim 6, further comprising a reverse transcriptase.

8. A method of amplifying a target nucleic acid, the method comprising:
providing a sample; and
mixing the sample, or a portion thereof, with a composition as in claim 1 to enable amplification of the target nucleic acid.

9. The method of claim 8, wherein amplification of the target nucleic acid comprises a LAMP or LAMP-like isothermal amplification reaction.

10. The method of claim 8, wherein amplification of the target nucleic acid comprises a transduction reaction, the transduction reaction functioning to generate a transduction product when the target nucleic acid is present within the sample, and the transduction product functioning to modulate a downstream amplification reaction.

11. The method of claim 10, wherein the downstream amplification reaction is a LAMP reaction or other LAMP-like isothermal amplification reaction, and wherein the transduction product functions as a required primer for the downstream amplification reaction.

12. A composition for isothermal amplification of a target nucleic acid, the composition comprising:
a set of oligonucleotide primers configured to enable a transduction reaction that generates a transduction product when the target nucleic acid is present;
a threshold complex comprising a dimer between a moderator strand and a transduction product complement,
the transduction product complement including a 5′ section and a 3′ section,
the moderator strand including (i) a section that is complementary to the 3′ section of the transduction product complement and (ii) a 3′ modification that prevents polymerase extension of the moderator strand,
wherein when the moderator strand hybridizes to the transduction product complement, the 3′ section of the transduction product complement becomes sequestered while the 5′ section of the transduction product complement can freely hybridize with the transduction product, and
wherein release of the moderator strand from the threshold complex modulates a downstream amplification reaction.

13. The composition of claim 12, wherein the downstream amplification reaction comprises a LAMP or LAMP-like isothermal amplification reaction.

14. The composition of claim 12, wherein the transduction product promotes the LAMP or LAMP-like isothermal amplification reaction, and wherein the moderator strand restrains the amplification reaction.

15. A method of amplifying nucleic acid using a thresholding mechanism, the method comprising:
providing a sample; and
mixing the sample, or a portion thereof, with a composition as in claim 12 to enable amplification of a target nucleic acid in the transduction reaction,
wherein the transduction reaction generates the transduction product, and wherein the threshold complex limits activation of the downstream amplification reaction by the transduction product until sufficient transduction product is generated.

16. A composition for isothermal amplification of a target nucleic acid, the composition comprising:
a first set of oligonucleotide primers configured to enable a transduction reaction that generates a transduction product, in the form of a reactivation strand, when the target nucleic acid is present;
a second set of oligonucleotide primers configured to enable a downstream amplification reaction; and
a deactivation strand annealed with an activatable primer of the second set of primers to thereby deactivate the activatable primer, the deactivation strand including (i) a complementary section hybridized with at least a portion of the activatable primer and (ii) a 3′ overhang,
wherein the reactivation strand is complementary to the deactivation strand such that the activatable primer is released from the deactivation strand upon hybridization of the reactivation strand and the deactivation strand.

17. The composition of claim 16, wherein the downstream amplification reaction utilizes a universal template.

18. The composition of claim 16, wherein the reactivation strand includes a 3′ overhang when hybridized with the deactivation strand.

19. The composition of claim 16, wherein the downstream amplification reaction is a LAMP or LAMP-like isothermal amplification reaction.

20. A method of amplifying nucleic acid by reactivating a deactivated primer, the method comprising:
providing a sample; and
mixing the sample, or a portion or extract thereof, with a composition as in claim 16 to enable amplification of a target nucleic acid in the transduction reaction,
wherein the transduction reaction generates the reactivation strand and wherein the reactivation strand hybridizes with the deactivation strand to release the activatable primer to thereby enable the downstream amplification reaction.
